(19) United States
(12) Patent Application Publication
(10) Pub. No.: US 2011/0172582 A1
(43) Pub. Date: Jul. 14, 2011

(54) WOUND DRESSING RETAINER

(76) Inventor: Michael Eric Darian, Ambler, PA (US)

(21) Appl. No.: 12/821,821

(22) Filed: Jun. 23, 2010

Related U.S. Application Data
(60) Provisional application No. 61/293,945, filed on Jan. 11, 2010.

(51) Int. Cl. A61F 15/00

(52) U.S. Cl. 602/79

(57) ABSTRACT

A wound dressing retainer is composed of a layer of flexible sheet material having a first surface for adhesion to a patient's skin, and a second surface having a plurality of oblique barbs for penetrating interstices of a gauze bandage, and thereby retaining the gauze bandage against sliding movement.

### CROSS-REFERENCE TO RELATED APPLICATION

This application claims priority on the basis of provisional application 61/293,945, filed on Jan. 11, 2010.

### FIELD OF THE INVENTION

The invention relates generally to medical devices, and more particularly to a novel retainer for preventing a gauze bandage or similar wound dressing from sliding on a patient's skin.

### BACKGROUND OF THE INVENTION

A gauze bandage is commonly used as a wound dressing. When applied to a patient's arm or leg, a strip of gauze having length equal to at least several times the circumference of the limb to which it is to be applied is wound around the limb, and either split and tied, or secured by means of adhesive tape. When adhesive tape is used to secure gauze wound around a patient's limb, a small length of tape is attached to an outer end of the strip of the gauze strip and to an adjacent portion of the outer winding of the strip.

A gauze strip, whether tied to itself or taped to itself, tends to move by gravity or inertia, necessitating frequent repositioning of the dressing over the wound. Undesired movements of the gauze dressing can be avoided by securing the dressing to the patient's skin using adhesive tape. However, whenever the dressing is changed, the tape must be removed from the patient's skin. Removal of the tape pulls out hair, causing pain and irritation. An alternative solution is to surround the bandage with an elastic sleeve. An elastic sleeve, however, can inhibit blood circulation.

### SUMMARY OF THE INVENTION

This invention is a device which can secure a wound dressing in order to avoid displacement resulting from gravity or inertia, and which does not need to be removed when the dressing is changed. Briefly, the device is composed of a sheet of material having an adhesive-coated face that can adhere to a patient's skin, and a set of barbs protruding obliquely from the opposite face for engaging a layer of gauze in a gauze bandage to prevent the gauze from sliding.

More particularly, the dressing retainer according to the invention comprises a layer of sheet material having a first surface adapted to face in a first direction toward a patient's skin, an adhesive coating on said first surface for securing the layer of sheet material to a patient's skin, and a plurality of barbs secured to a second surface of the layer of sheet material. The second surface is parallel to the first surface and faces in a second direction opposite to the first direction. Each of the barbs protrudes away from the second surface in a direction oblique with respect to first and second surfaces. The barbs are preferably tapered so that they can pass between adjacent yarns in a gauze bandage and prevent the gauze bandage from moving by gravity or inertia in one direction parallel to the first and second surfaces, while allowing the gauze bandage to be disconnected from the device by movement of the gauze bandage in an opposite direction. Preferably all of the barbs protrude in the same direction.

In a preferred embodiment, the barbs are composed of a synthetic resin having a rigidity higher than that of the layer of sheet material.

In a preferred embodiment, the barbs are unitary parts of plural grip elements secured to the second surface of the sheet material. In this preferred embodiment, the layer of sheet material is more flexible than the material of the grip elements, and the grip element are separate from one another. As a result a flexible area of the layer of sheet material is disposed behind a space between each grip element and an adjacent grip element, and the retainer can be bent more easily to conform to the shape of the skin or other surface to which it is to be attached despite the relatively high rigidity of the grip material. In this embodiment, each of the grip elements can be formed so that it has only a single barb.

Each barb is preferably a substantially triangular element formed by punching a triangular opening in a grip element. Each barb has a base connected to a grip element and an apex pointing in the oblique third direction. The apex can be rounded to avoid injury to the user while still being capable of penetrating a gauze bandage and securing it against sliding movement.

Because the barbs can be small in size and difficult to see, it is desirable to provide a feature by which the user can easily determine the direction of the barbs. Markings can be provided on the second surface of the layer of sheet material. However, a still better alternative is to form the layer of sheet material in an asymmetric shape so that it has a tab protruding in the direction in which the gauze must be moved to release it from the barbs. The tab can also be gripped by the user, facilitating removal of the retainer from the skin.

In a preferred embodiment, the barbs are unitary parts of plural grip elements secured to the second surface of the layer of sheet material, each of the grip elements includes only a single barb, and the plural grip elements are disposed on the second surface in a two-dimensional array.

The invention can also be thought of as a wound dressing including a gauze bandage as one of its elements, the gauze bandage being secured to the retainer as explained above and prevented from being moved by gravity or inertia in one direction but allowed to be disconnected from the retainer by movement in an opposite direction.

### BRIEF DESCRIPTION OF THE DRAWINGS

FIG. 1 is an elevational view of the wound dressing retainer of the invention, as seen from the side on which the barbs are attached;

FIG. 2 is an elevational view, similar to FIG. 1, of a modified version of the wound dressing retainer;

FIG. 3 is an elevational view, of another modified version of the wound dressing retainer;

FIG. 4 is a schematic elevational view showing the retainer attached to an individual's skin in proximity to a wound to which a gauze bandage is about to be applied;

FIG. 5 is a schematic elevational view showing the same individual with the gauze bandage applied;

FIG. 6 is a cross-sectional view through a retainer and a part of a gauze bandage attached thereto;

FIG. 7 is a rear elevational view of a wound dressing retainer of the invention, as seen from the side opposite from the side on which the barbs are attached, showing an adhesive; and

FIG. 8 is an elevational view similar to FIG. 7, showing a modified adhesive pattern.

### DETAILED DESCRIPTION OF THE PREFERRED EMBODIMENTS

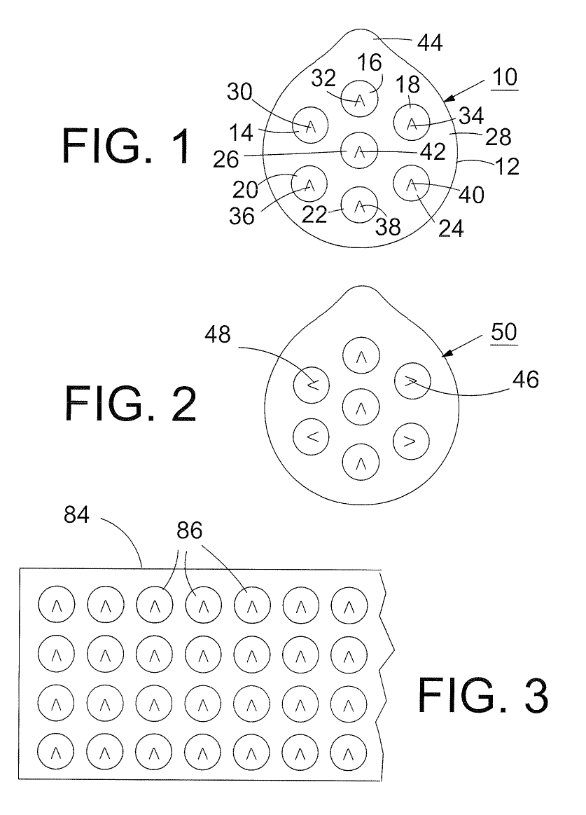

As shown in FIG. 1, a preferred retainer 10 according to the invention comprises a layer 12 of sheet material, preferably a foam, and a two-dimensional array of discrete grip elements 14-26 disposed on a front surface 28 of the sheet. The pattern of grip elements is preferably a regular pattern, and, in the embodiment shown, it consists of a central grip element surrounded by six more grip elements disposed in a circle. Alternatively a pattern consisting of rows and columns, or any of various other patterns can be utilized.

Each grip element is in the form of a flat, thin disc having a triangular barb protruding outwardly in a direction oblique with respect to the surface 28 of the sheet. The barbs, 30-42 preferably all protrude in the same direction, which is preferably upward. The retainer is preferably asymmetric, having a tab 44 which facilitates removal. The tab also extends in a direction corresponding to the direction in which the barbs extend obliquely, and thus provides a visual indication of the direction of the barbs. If the user attaches the retainer with the tab extending upward, the barbs will extend obliquely upward.

In the alternative version shown in FIG. 2, two of the barbs, 46 and 48 on retainer 50 extend laterally, to the left and right respectively, while the other tabs extend upward. In this case, the upwardly extending tabs prevent gauze from sliding downward, and the two laterally extending tabs assist in preventing the gauze from being dislodged from the retainer.

As shown in FIG. 7, the back surface 52 of the retainer 10, i.e., the surface opposite from the front surface 28 (FIG. 1), is provided with a layer 54 of adhesive, preferably a non-hardening adhesive suitable for contact with human skin and having a degree of adhesion that will firmly secure the sheet material to the skin but allow the sheet material to be peeled away from the skin without excessive difficulty. As supplied, the retainer should include a peelable release layer 55 over the adhesive layer to protect the adhesive until the retainer is ready for use.

In an alternative version of the retainer, the adhesive on the back side of the retainer can be provided in multiple, separate areas in order to make removal easier. For example, in FIG. 8, the back surface 56 of retainer 58 has four circular areas of adhesive, 60, 62, 64, and 66. This version should also have a peelable release layer (not shown).

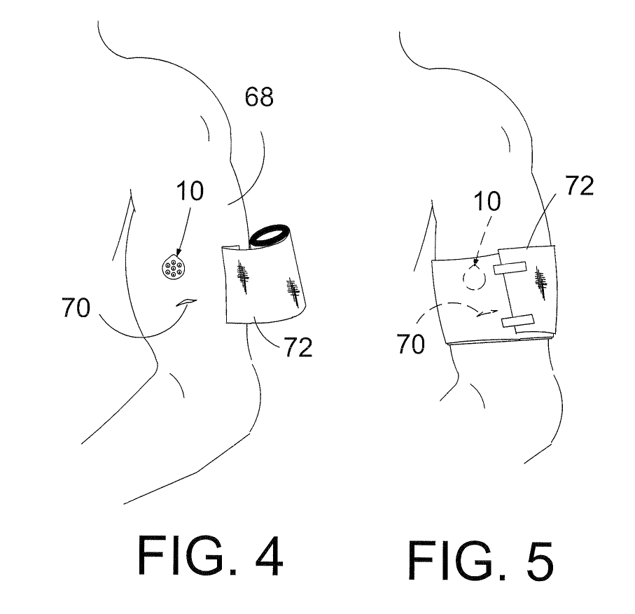

As shown in FIG. 4, retainer 10 is adhesively secured to the upper arm 68 of a patient a short distance above a wound 70 that is about to be bandaged by a rolled gauze bandage 72. That retainer should be positioned so that the bandage, when applied covers both the wound and the retainer as shown in FIG. 5.

In the case shown, where the wound is on the patient's arm it is customary to wind the gauze bandage around the arm in several layers and to secure the bandage to itself by means of adhesive tape. As mentioned previously, such a bandage has a tendency to slide downward by gravity, or as a result of inertia when the patient's arm is swung rapidly. The barbs of the retainer, however, grip the innermost layer of gauze and prevent the bandage from sliding.

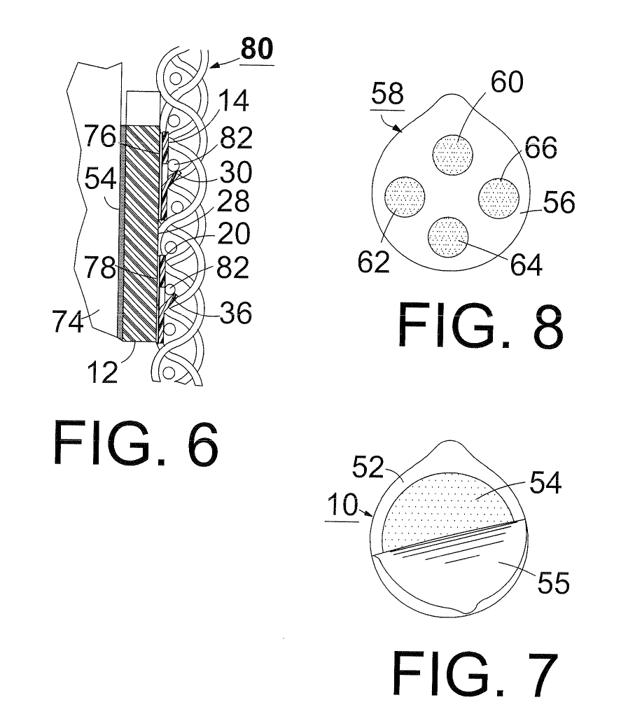

The manner in which the barbs cooperate with a layer of gauze is shown in FIG. 6, where the back face 52 of the sheet material layer 12 is attached to the patient's skin 74 by an adhesive layer 54, and grip members 14 and 20 are individually secured to the front face 28 of the sheet material layer by adhesive layers 76 and 78, respectively. The barbs 30 and 36 of grip elements 14 and 20 protrude through interstices formed by the warp and weft yarns of a layer 80 of the gauze bandage 72, catching weft yarns 82, and preventing the inner layer from sliding downward. As the several layers of the bandage are held together by friction, the entire bandage is prevented from sliding downward.

The gauze bandage can be removed by unwinding its several layers and disengaging the inner layer from the retainer. A new bandage can be applied without removal of the retainer. At an appropriate time, the retainer can be removed by peeling it away from the patient's skin. As shown in FIGS. 6 and 7, the area underneath the tab of the foam layer is free of adhesive, allowing it to be grasped easily for removal.

Although the retainer is especially advantageous when used with bandages wound around a patient's arm or leg, it can also be used to retain any of various other kinds of gauze bandages.

An example of a preferred material for the sheet layer is a closed cell polyvinyl chloride (PVC) foam tape having a thickness of 22.5 mils (0.6 mm), an adhesive layer capable of removably adhering the foam tape to human skin and a peelable protective liner over the adhesive layer. A suitable foam tape is 3M 9781 tape, available from 3M Company, 3M Center, St. Paul, Minn., 55144, U.S.A.

The barbed grips are preferably composed of a layer of polycarbonate resin having a thickness of 0.011 inch (0.28 mm).

The grips are adhered to the face of the foam tape opposite from the face on which the skin-contacting adhesive is disposed by an adhesive, preferably a high performance acrylic adhesive such as 3M 9482PC adhesive, also available from 3M Company.

In the preferred embodiment as described, the grip elements are discrete elements composed of synthetic resin, each having a single barb. The use of grip elements each having a single barb provides the retainer with a high degree of flexibiilty. Advantages of the invention can be realized in embodiments in which each of the grip elements has plural barbs, and even in embodiments in which the retainer comprises a flexible sheet having a single, unitary, grip element with multiple barbs, although retainers will have more limited flexibility.

The barbed grips can be composed of any of various alternative materials including metal and various resins other than polycarbonates.

The barbs can be of various sizes and shapes. The preferred barb extends from the plane of the disc portion of the grip element at an angle of about 30 degrees, and has a length of approximately 1.35 mm. The barbs are preferably generally triangular in shape with rounded tips. The barbs can have various alternative shapes such as semicircular shapes.

In a modified version of the invention, illustrated in FIG. 3, a sheet 84 of material similar to layer 12 is provided with a plurality of individual barbed grip elements 86 preferably in a regular pattern that enables the user to cut out a retainer of almost any desired size and shape. Here again, the back side of the sheet 84 is provided with a layer of adhesive, or with plural discrete adhesive areas for attachment to a patient's skin. As supplied, the sheet is provided with a peelable release layer over the adhesive. The sheet can be supplied in various forms, for example, as a flat sheet, or as a roll

Many other modifications in materials, dimensions, configurations, and other aspects of the retainers and wound dressings described without departing from the scope of the invention as defined in the following claims.

What is claimed is:

1. A wound dressing retainer comprising:
a layer of sheet material having a first surface adapted to face in a first direction toward a patient's skin;
an adhesive coating on said first surface for securing said layer of sheet material to a patient's skin;
a plurality of barbs secured to a second surface of said layer of sheet material, said second surface being parallel to the first surface and facing in a second direction opposite to said first direction, and each of said barbs protruding away from the second surface of said layer in a direction oblique with respect to said first and second surfaces, said barbs being tapered whereby they can pass between adjacent yarns in a gauze bandage and prevent the gauze bandage from moving by gravity or inertia in one direction parallel to said first and second surfaces, while allowing the gauze bandage to be disconnected from the device by movement of the gauze bandage in a direction opposite to said one direction.

2. A wound dressing retainer according to claim 1, in which all of said barbs protrude in the same direction.

3. A wound dressing retainer according to claim 1, in which said barbs are composed of a synthetic resin having a higher rigidity than said layer of sheet material.

4. A wound dressing retainer according to claim 3, in which said barbs are unitary parts of plural grip elements secured to said second surface, in which the material of said layer of sheet material is more flexible that the material of said grip elements, and in which said grip element are separate from one another whereby a flexible area of said layer of sheet material is disposed behind a space between each grip element and an adjacent one of said grip elements.

5. A wound dressing retainer according to claim 1, in which said barbs are unitary parts of plural grip elements secured to said second surface, in which the material of said layer of sheet material is more flexible that the material of said grip elements, and in which said grip element are separate from one another whereby a flexible area of said layer of sheet material is disposed behind a space between each grip element and an adjacent one of said grip elements.

6. A wound dressing retainer according to claim 5, in which each of said grip elements includes only a single barb.

7. A wound dressing retainer according to claim 1, in which each of said barbs is a unitary part of a grip element composed of a rigid synthetic resin having a higher rigidity than that of said layer of sheet material, and in which each said barb is a substantially triangular element having a base connected to a grip element and an apex pointing in said third direction, and formed by punching a triangular opening in a grip element.

8. A wound dressing retainer according to claim 7, in which the apex of each barb is rounded.

9. A wound dressing retainer according to claim 1, in which the layer of sheet material is shaped to form a tab extending in said direction opposite to said one direction.

10. A wound dressing retainer according to claim 1, in which said barbs are unitary parts of plural grip elements secured to said second surface, in which each of said grip elements includes only a single barb, and in which the plural grip elements are disposed on said second surface in a two-dimensional array.

11. A wound dressing comprising:
a gauze bandage;
a layer of sheet material having a first surface facing in a first direction toward, said first surface being attached to a patient's skin;
a plurality of barbs secured to a second surface of said layer of sheet material, said second surface being parallel to the first surface and facing in a second direction opposite to said first direction, and each of said barbs protruding away from the second surface of said layer in a third direction oblique with respect to first and second surfaces, said barbs extending between adjacent yarns in said gauze bandage and preventing the gauze bandage from moving by gravity or inertia in one direction parallel to said first and second surfaces, while allowing the gauze bandage to be disconnected from the device by movement of the gauze bandage in a direction opposite to said one direction.

12. A wound dressing according to claim 11, including an adhesive coating on said first surface, said adhesive coating securing said layer of sheet material to said patient's skin.

13. A wound dressing according to claim 11, in which all of said barbs protrude in the same direction.

14. A wound dressing according to claim 11, in which said barbs are tapered so that their widths decrease proceeding in said third direction.

15. A wound dressing retainer according to claim 14, in which said barbs are composed of a synthetic resin having a higher rigidity than said layer of sheet material.

16. A wound dressing retainer according to claim 15, in which said barbs are unitary parts of plural grip elements secured to said second surface, in which the material of said layer of sheet material is more flexible that the material of said grip elements, and in which said grip element are separate from one another whereby a flexible area of said layer of sheet material is disposed behind a space between each grip element and an adjacent one of said grip elements.

17. A wound dressing retainer according to claim 14, in which said barbs are unitary parts of plural grip elements secured to said second surface, in which the material of said layer of sheet material is more flexible that the material of said grip elements, and in which said grip element are separate from one another whereby a flexible area of said layer of sheet material is disposed behind a space between each grip element and an adjacent one of said grip elements.

18. A wound dressing retainer according to claim 17, in which each of said grip elements includes only a single barb.

19. A wound dressing retainer according to claim 14, in which each of said barbs is a unitary part of a grip element composed of a rigid synthetic resin having a higher rigidity than that of said layer of sheet material, and in which each said barb is a substantially triangular element having a base connected to a grip element and an apex pointing in said third direction, and formed by punching a triangular opening in a grip element.

20. A wound dressing retainer according to claim 19, in which the apex of each barb is rounded.
